SEQUENCE LISTING (3 sequences)

SEQ ID NO: 1 - DNA, 2181 bp, Homo sapiens
atggcagtgggtgagcccctggtgcacatcagggtcactcttctgctgctctggtttgggatgtttttgtctatttctggccactctcaggccaggccctcccagtatttcacttctccagaagtggtgatccctttgaaggtgatcagcaggggcagaggtgcaaaggctcctggatggctctcctatagcctgcggtttgggggacagagatacattgtccacatgagggtaaataagctgttgtttgctgcacaccttcctgtgttcacctacacagagcagcatgccctgctccaggatcagcccttcatccaggatgactgctactaccatggttatgtggagggggtccctgagtccttggttgcccttagtacctgttctgggggctttcttggaatgctacagataaatgaccttgtttatgaaatcaagccaattagtgtttctgccacatttgaacacctagtatataagatagacagtgatgatacacagtttccacctatgagatgtgggttaacagaagagaaaatagcacaccagatggagttgcaattgtcatataatttcactctgaagcaaagttcttttgtgggctggtggacccatcagcggtttgttgagctggtagtggtcgtggataatattagatatcttttctctcaaagtaatgcaacaacagtgcagcatgaagtatttaacgttgtcaatatagtggattccttctatcatcctttggaggttgatgtaattttgactggaattgatatatggactgcatcaaatccacttcctaccagtggagacctagataatgttttagaggacttttctatttggaagaattataaccttaataatcgactacaacatgatgttgcacatcttttcataaaagacacacaaggcatgaagcttggtgttgcctatgttaaaggaatatgccagaatccttttaatactggagttgatgtttttgaagacaacaggttggtcgtttttgcaattactttgggccacgagcttggtcataatttgggtatgcaacatgacacccagtggtgtgtgtgcgagctacagtggtgcataatgcatgcctatagaaaggtgacaactaaatttagcaactgcagttatgcccaatattgggacagtactatcagtagtggattatgtattcaaccgcctccatatccagggaatatatttagactgaagtactgtgggaatctagtggttgaagaaggggaggaatgtgactgtggaaccatacggcagtgtgcaaaagatccctgttgtctgttaaactgtactctacatcctggggctgcttgtgcttttggaatatgttgcaaagactgcaaatttctgccatcaggaactttatgtagacaacaagttggtgaatgtgaccttccagagtggtgcaatgggacatcccatcaatgcccagatgatgtgtatgtgcaggacgggatctcctgtaatgtgaatgccttctgctatgaaaagacgtgtaataaccatgatatacaatgtaaagagatttttggccaagatgcaaggagtgcatctcagagttgctaccaagaaatcaacacccaaggaaaccgtttcggtcactgtggtattgtaggcacaacatatgtaaaatgttggacccctgatatcatgtgtgggagggttcagtgtgaaaatgtgggagtaattcccaatctgatagagcattctacagtgcagcagtttcacctcaatgacaccacttgctggggcactgattatcatttagggatggctatacctgatattggtgaggtgaaagatggcacagtatgtggtccagaaaagatctgcatccgtaagaagtgtgccagtatggttcatctgtcacaagcctgtcagcctaagacctgcaacatgaggggaatctgcaacaacaaacaacactgtcactgcaaccatgaatgggcacccccatactgcaaggacaaaggctatggaggtagtgctgatagtggcccacctcctaagaacaacatggaaggattaaatgtgatgggaaagttgcgttacctgtcactattgtgccttcttcctttggttgcttttttattattttgcttacatgtgctttttaagaaacgcacaaaaagtaaagaagatgaagaaggataa

SEQ ID NO: 2 - protein, 726 aa, Homo sapiens
MAVGEPLVHIRVTLLLLWFGMFLSISGHSQARPSQYFTSPEVVIPLKVISRGRGAKAPGWLSYSLRFGGQRYIVHMRVNKLLFAAHLPVFTYTEQHALLQDQPFIQDDCYYHGYVEGVPESLVALSTCSGGFLGMLQINDLVYEIKPISVSATFEHLVYKIDSDDTQFPPMRCGLTEEKIAHQMELQLSYNFTLKQSSFVGWWTHQRFVELVVVVDNIRYLFSQSNATTVQHEVFNVVNIVDSFYHPLEVDVILTGIDIWTASNPLPTSGDLDNVLEDFSIWKNYNLNNRLQHDVAHLFIKDTQGMKLGVAYVKGICQNPFNTGVDVFEDNRLVVFAITLGHELGHNLGMQHDTQWCVCELQWCIMHAYRKVTTKFSNCSYAQYWDSTISSGLCIQPPPYPGNIFRLKYCGNLVVEEGEECDCGTIRQCAKDPCCLLNCTLHPGAACAFGICCKDCKFLPSGTLCRQQVGECDLPEWCNGTSHQCPDDVYVQDGISCNVNAFCYEKTCNNHDIQCKEIFGQDARSASQSCYQEINTQGNRFGHCGIVGTTYVKCWTPDIMCGRVQCENVGVIPNLIEHSTVQQFHLNDTTCWGTDYHLGMAIPDIGEVKDGTVCGPEKICIRKKCASMVHLSQACQPKTCNMRGICNNKQHCHCNHEWAPPYCKDKGYGGSADSGPPPKNNMEGLNVMGKLRYLSLLCLLPLVAFLLFCLHVLFKKRTKSKEDEEG

SEQ ID NO: 3 - protein, 535 aa, Homo sapiens
METDTLLLWVLLLWVPGSTGTSCGNLVVEEGEECDCGTIRQCAKDPCCLLNCTLHPGAACAFGICCKDCKFLPSGTLCRQQVGECDLPEWCNGTSHQCPDDVYVQDGISCNVNAFCYEKTCNNHDIQCKEIFGQDARSASQSCYQEINTQGNRFGHCGIVGTTYVKCWTPDIMCGRVQCENVGVIPNLIEHSTVQQFHLNDTTCWGTDYHLGMAIPDIGEVKDGTVCGPEIICIRKKCASMVHLSQACQPKTCNMRGICNNKQHCHCNHEWAPPYCKDKGYGGSADSGPPPKNNMEGLNVMGKLRGSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2009/0017492 A1
(43) Pub. Date: Jan. 15, 2009

(54) SVPH1-26 DNA and polypeptides

(75) Inventor: Douglas Pat Cerretti, Shoreline, WA (US)

Correspondence Address: IMMUNEX CORPORATION;LAW DEPARTMENT, 1201 AMGEN COURT WEST, SEATTLE, WA, 98119, US

(73) Assignee: Immunex Corporation, Thousand Oaks, CA (US)

(21) Appl. No.: 11/818,872

(22) Filed: Jun. 15, 2007

Related U.S. Application Data
(63) Continuation of application No. 10/633,202, filed on Jul. 29, 2003, now abandoned.
Application No. 10/633,202 is a continuation of application No. 09/561,779, filed on May 1, 2000, now abandoned.
Application No. 09/561,779 is a continuation of application No. PCT/US98/22965, filed on Oct. 30, 1998.
(60) Provisional application No. 60/063,571, filed on Oct. 30, 1997.

(51) Int. Cl. C12P 21/00; C07H 21/00; C07K 16/00; C12N 15/63

(52) U.S. Cl. 435/69.1; 536/23.2; 536/23.5; 435/320.1; 530/387.9

(57) ABSTRACT

DNA encoding SVPH1-26 polypeptides and methods for using the encoded proteinase and polypeptides are disclosed. SVPH1-26 is expressed in testis.

### FIELD OF THE INVENTION

The invention is directed to purified and isolated SVPH1-26 polypeptides, the nucleic acids encoding such polypeptides, processes for production of recombinant forms of such polypeptides, antibodies generated against these polypeptides, fragmented peptides derived from these polypeptides, the use of such polypeptides and fragmented peptides as molecular weight markers, the use of such polypeptides and fragmented peptides as controls for peptide fragmentation, the use of such nucleic acids, polypeptides, and antibodies as cell and tissue markers, and kits comprising these reagents.

### BACKGROUND OF THE INVENTION

The discovery and identification of proteins is at the forefront of modern molecular biology and biochemistry. The identification of the primary structure, or sequence, of a sample protein is the culmination of an arduous process of experimentation. In order to identify an unknown sample protein, the investigator can rely upon comparison of the unknown sample protein to known peptides using a variety of techniques known to those skilled in the art. For instance, proteins are routinely analyzed using techniques such as electrophoresis, sedimentation, chromatography, and mass spectrometry.

Comparison of an unknown protein sample to polypeptides of known molecular weight allows a determination of the apparent molecular weight of the unknown protein sample (T. D. Brock and M. T. Madigan, Biology of Microorganisms 76-77 (Prentice Hall, 6d ed. 1991)). Protein molecular weight standards are commercially available to assist in the estimation of molecular weights of unknown protein samples (New England Biolabs Inc. Catalog:130-131, 1995; J. L. Hartley, U.S. Pat. No. 5,449,758). However, the molecular weight standards may not correspond closely enough in size to the unknown sample protein to allow an accurate estimation of apparent molecular weight.

The difficulty in estimation of molecular weight is compounded in the case of proteins that are subjected to fragmentation by chemical or enzymatic means (A. L. Lehninger, Biochemistry 106-108 (Worth Books, 2d ed. 1981)). Chemical fragmentation can be achieved by incubation of a protein with a chemical, such as cyanogen bromide, which leads to cleavage of the peptide bond on the carboxyl side of methionine residues (E. Gross, Methods in Enz. 11:238-255, 1967). Enzymatic fragmentation of a protein can be achieved by incubation of a protein with a protease that cleaves at multiple amino acid residues (D. W. Cleveland et al., J. Biol. Chem. 252:1102-1106, 1977). Enzymatic fragmentation of a protein can also be achieved by incubation of a protein with a protease, such as Achromobacter protease I (F. Sakiyama and A. Nakata, U.S. Pat. No. 5,248,599; T. Masaki et al., Biochim. Biophys. Acta 660:44-50, 1981; T. Masaki et al., Biochim. Biophys. Acta 660:51-55, 1981), which leads to cleavage of the peptide bond on the carboxyl side of lysine residues. The molecular weights of the fragmented peptides can cover a large range of molecular weights and the peptides can be numerous. Variations in the degree of fragmentation can also be accomplished (D. W. Cleveland et al., J. Biol. Chem. 252:1102-1106, 1977).

The unique nature of the composition of a protein with regard to its specific amino acid constituents results in a unique positioning of cleavage sites within the protein. Specific fragmentation of a protein by chemical or enzymatic cleavage results in a unique “peptide fingerprint” (D. W. Cleveland et al., J. Biol. Chem. 252:1102-1106, 1977; M. Brown et al., J. Gen. Virol. 50:309-316, 1980). Consequently, cleavage at specific sites results in reproducible fragmentation of a given protein into peptides of precise molecular weights. Furthermore, these peptides possess unique charge characteristics that determine the isoelectric pH of the peptide. These unique characteristics can be exploited using a variety of electrophoretic and other techniques (T. D. Brock and M. T. Madigan, Biology of Microorganisms 76-77 (Prentice Hall, 6d ed. 1991)).

When a peptide fingerprint of an unknown protein is obtained, this can be compared to a database of known proteins to assist in the identification of the unknown protein (W. J. Henzel et al., Proc. Natl. Acad. Sci. USA 90:5011-5015, 1993; B. Thiede et al., Electrophoresis 1996, 17:588-599, 1996). A variety of computer software programs are accessible via the Internet to the skilled artisan for the facilitation of such comparisons, such as MultiIdent (Internet site: www.expasy.ch/sprot/multiident.html), PeptideSearch (Internet site: www.mann.embl-heiedelberg.de . . . deSearch/FR_PeptideSearchForm.html), and ProFound (Internet site: www.chait-sgi.rockefeller.edu/cgi-bin/prot-id-frag.html). These programs allow the user to specify the cleavage agent and the molecular weights of the fragmented peptides within a designated tolerance. The programs compare these molecular weights to protein databases to assist in the elucidation of the identity of the sample protein. Accurate information concerning the number of fragmented peptides and the precise molecular weight of those peptides is required for accurate identification. Therefore, increasing the accuracy in the determination of the number of fragmented peptides and the precise molecular weight of those peptides should result in enhanced success in the identification of unknown proteins.

Fragmentation of proteins is further employed for the production of fragments for amino acid composition analysis and protein sequencing (P. Matsudiara, J. Biol. Chem. 262:10035-10038, 1987; C. Eckerskorn et al., Electrophoresis 1988, 9:830-838, 1988), particularly the production of fragments from proteins with a “blocked” N-terminus. In addition, fragmentation of proteins can be used in the preparation of peptides for mass spectrometry (W. J. Henzel et al., Proc. Natl. Acad. Sci. USA 90:5011-5015, 1993; B. Thiede et al., Electrophoresis 1996, 17:588-599, 1996), for immunization, for affinity selection (R. A. Brown, U.S. Pat. No. 5,151,412), for determination of modification sites (e.g. phosphorylation), for generation of active biological compounds (T. D. Brock and M. T. Madigan, Biology of Microorganisms 300-301 (Prentice Hall, 6d ed. 1991)), and for differentiation of homologous proteins (M. Brown et al., Gen. Virol. 50:309-316, 1980).

In view of the continuing interest in protein research and the elucidation of protein structure and properties, there exists a need in the art for polypeptides suitable for use in peptide fragmentation studies and in molecular weight measurements.

### SUMMARY OF THE INVENTION

The invention aids in fulfilling this need in the art. The invention encompasses an isolated nucleic acid molecule comprising the DNA sequence of SEQ ID NO:1 and an isolated nucleic acid molecule encoding the amino acid sequence of SEQ ID NO:2. The invention also encompasses nucleic acid molecules complementary to these sequences. As such, the invention includes double-stranded nucleic acid sequences comprising the DNA sequence of SEQ ID NO:1 and isolated nucleic acid molecules encoding the amino acid sequence of SEQ ID NO:2. Both single-stranded and double-stranded RNA and DNA SVPH1-26 nucleic acid molecules are encompassed by the invention. These molecules can be used to detect both single-stranded and double-stranded RNA and DNA variants of SVPH1-26 encompassed by the invention. A double-stranded DNA probe allows the detection of nucleic acid molecules equivalent to either strand of the nucleic acid molecule. Isolated nucleic acid molecules that hybridize to a denatured, double-stranded DNA comprising the DNA sequence of SEQ ID NO:1 or an isolated nucleic acid molecule encoding the amino acid sequence of SEQ ID NO:2 under conditions of moderate stringency in 50% formamide and 6×SSC, at 42° C. with washing conditions of 60° C., 0.5×SSC, 0.1% SDS are encompassed by the invention.

The invention further encompasses isolated nucleic acid molecules derived by in vitro mutagenesis from SEQ ID NO:1. In vitro mutagenesis would include numerous techniques known in the art including, but not limited to, site-directed mutagenesis, random mutagenesis, and in vitro nucleic acid synthesis. The invention also encompasses isolated nucleic acid molecules degenerate from SEQ ID NO:1 as a result of the genetic code, isolated nucleic acid molecules which are allelic variants of human SVPH1-26 DNA or a species homolog of SVPH1-26 DNA. The invention also encompasses recombinant vectors that direct the expression of these nucleic acid molecules and host cells transformed or transfected with these vectors.

The invention also encompasses isolated polypeptides encoded by these nucleic acid molecules, including isolated polypeptides having a molecular weight of approximately 82 kD as determined by SDS-PAGE and isolated polypeptides in non-glycosylated form. Isolated polyclonal or monoclonal antibodies that bind to these polypeptides are encompassed by the invention. The invention further encompasses methods for the production of SVPH1-26 polypeptides including culturing a host cell under conditions promoting expression and recovering the polypeptide from the culture medium. Especially, the expression of SVPH1-26 polypeptides in bacteria, yeast, plant, and animal cells is encompassed by the invention.

In addition, assays utilizing SVPH1-26 polypeptides to screen for potential inhibitors of activity associated with SVPH1-26 polypeptide counter-structure molecules, and methods of using SVPH1-26 polypeptides as therapeutic agents for the treatment of diseases mediated by SVPH1-26 polypeptide counter-structure molecules are encompassed by the invention. Further, methods of using SVPH1-26 polypeptides in the design of inhibitors thereof are also an aspect of the invention.

The invention further encompasses the fragmented peptides produced from SVPH1-26 polypeptides by chemical or enzymatic treatment. In addition, forms of SVPH1-26 polypeptide molecular weight markers and fragmented peptides thereof, wherein at least one of the sites necessary for fragmentation by chemical or enzymatic means has been mutated, are an aspect of the invention.

The invention also encompasses a method for the visualization of SVPH1-26 polypeptide molecular weight markers and fragmented peptides thereof using electrophoresis. The invention further includes a method for using SVPH1-26 polypeptide molecular weight markers and fragmented peptides thereof as molecular weight markers that allow the estimation of the molecular weight of a protein or a fragmented protein sample. The invention further encompasses methods for using SVPH1-26 polypeptides and fragmented peptides thereof as markers, which aid in the determination of the isoelectric point of a sample protein. The invention also encompasses methods for using SVPH1-26 polypeptides and fragmented peptides thereof as controls for establishing the extent of fragmentation of a protein sample.

Further encompassed by this invention are kits to aid the determination of molecular weights of a sample protein utilizing SVPH1-26 polypeptide molecular weight markers, fragmented peptides thereof, and forms of SVPH1-26 polypeptide molecular weight markers, wherein at least one of the sites necessary for fragmentation by chemical or enzymatic means has been mutated.

Further encompassed by this invention are methods of using SVPH1-26 nucleic acids, polypeptides, and antibodies as cell and tissue markers in the identification and purification of SVPH1-26 expressing cells.

### BRIEF DESCRIPTION OF THE DRAWINGS

This invention will be more fully described with reference to the drawings in which:

FIG. 1 is the nucleotide sequence of SVPH1-26 DNA, SEQ ID NO:1.

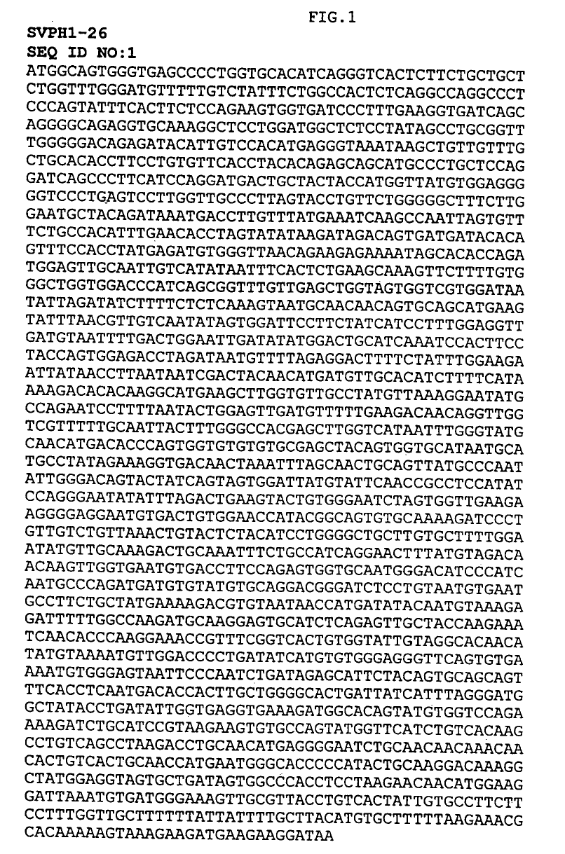

FIG. 2 is the amino acid sequence of SVPH1-26 polypeptide, SEQ ID NO:2.

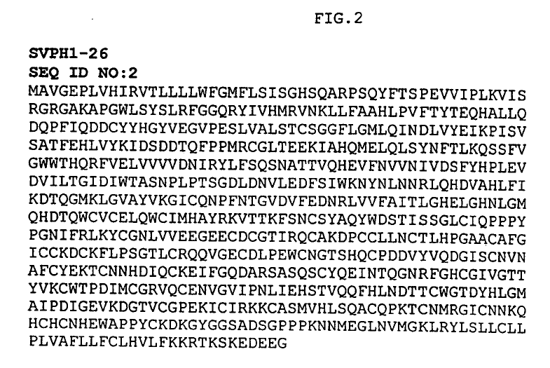

### DETAILED DESCRIPTION OF THE INVENTION

A cDNA encoding human SVPH1-26 polypeptide has been isolated and is disclosed in SEQ ID NO:1. This discovery of the cDNA encoding human SVPH1-26 polypeptide enables construction of expression vectors comprising nucleic acid sequences encoding SVPH1-26 polypeptides; host cells transfected or transformed with the expression vectors; biologically active human SVPH1-26 proteinase and SVPH1-26 molecular weight markers as isolated and purified proteins; and antibodies immunoreactive with SVPH1-26 polypeptides.

SVPH1-26 polypeptide (SEQ ID NO:2) has all of the conserved domain structures found in mammalian adamalysins (ADAMS): signal sequence (amino acids 1-27 of SEQ ID:2), pro domain (amino acids 28-197 of SEQ ID:2), catalytic domain including the three conserved His residues (amino acids 198-398 of SEQ ID:2), disintegrin domain (amino acids 399-502 of SEQ ID:2), Cys-rich domain (amino acids 503-692 of SEQ ID:2), transmembrane domain (amino acids 693-714 of SEQ ID:2), and a cytoplasmic domain (amino acids 715-726 of SEQ ID:2).

ADAMS 1-6 have been implicated in fertilization and/or spermatogenesis (Barker, H. L., Perry, A. C., Jones, R., and Hall, L., Biochim Biophys Acta, 1218, 429-31, 1994; Blobel, C. P., Wolfsberg, T. G., Turck, C. W., Myles, D. G., Primakoff, P., and White, J. M., Nature, 356, 248-252, 1992; Evans, J. P., Schultz, R. M., and Kopf, G. S., J. Cell Sci, 108, 3267-3278, 1995; Perry, A. C., Barker, H. L., Jones, R., and Hall., L., Biochim Biophsy Acta, 1207, 134-137, 1994; Perry, A. C., Gichuhi, P. M., Jones, R., and Hall, L., Biochem J, 307, 843-850, 1995; Perry, A. C., Jones, R., and Hall, L., Biochem J., 312, 239-244, 1995; Wolfsberg, T. G., Bazan, J. F., Blobel, C. P., Mules, D. G., Primakoff, P., and White, J. M., Proc Natl Acad Sci USA, 90, 10783-10787, 1993; and Wolfsberg, T. G., Straight, P. D., Gerena, R. L., Huovila, A. P., Primakoff, P., Myles, D. G., and White, J. M., Dev Biol, 169, 378-383, 1995). The finding that SVPH1-26 is specifically expressed in testis by Northern analysis also implicates this family member in fertilization and/or spermatogenesis. In addition, while ADAM1 has been found to be required for the fusion of sperm and egg, humans do not have an active form of this gene. Thus SVPH1-26 may be the human equivalent. The SVPH1-26 catalytic domain is required for biological activity. A proteinase inhibitor of the catalytic domain would inhibit SVPH1-26 activity and would be useful as a method for birth control. Also, an inhibitor of the disintegrin domain of SVPH1-26 may affect fertilization.

SVPH1-26 proteinase is a member of the snake venom protease family. SVPH1-26 proteinase is homologous to the TACE protein, with an amino acid identity of 20%. TACE is a proteinase required for the shedding of membrane proteins including TNF a, p80 TNFR, p60TNFR, L-selectin, type II IL-1R, and b-amyloid precursor protein. SVPH1-26 proteinase also shows homology with fertilin-a (35% amino acid homology), which is required for binding of sperm to egg; meltrin-a (33% amino acid homology), which is required for the fusion of myoblasts into muscle cells; reprolysin (24% amino acid homology), which cleaves myelin basic protein; and kuzbanian which is a Drosophila homologue of reprolysin which is required for neurogenesis and axonal extension. The proteinase activity of SVPH1-26 is likely involved in the shedding of membrane proteins.

In one embodiment of this invention, the expression of recombinant SVPH1-26 polypeptides can be accomplished utilizing fusion of sequences encoding SVPH1-26 polypeptides to sequences encoding another polypeptide to aid in the purification of SVPH1-26 polypeptides. An example of such a fusion is a fusion of sequences encoding a SVPH1-26 polypeptide to sequences encoding the product of the malE gene of the pMAL-c2 vector of New England Biolabs, Inc. Such a fusion allows for affinity purification of the fusion protein, as well as separation of the maltose binding protein portion of the fusion protein from the SVPH1-26 polypeptide after purification. It is understood of course that many different vectors and techniques can be used for the expression and purification of SVPH1-26 polypeptides and that this embodiment in no way limits the scope of the invention.

The insertion of DNA encoding the SVPH1-26 polypeptide into the pMAL-c2 vector can be accomplished in a variety of ways using known molecular biology techniques. The preferred construction of the insertion contains a termination codon adjoining the carboxyl terminal codon of the SVPH1-26 polypeptide. In addition, the preferred construction of the insertion results in the fusion of the amino terminus of the SVPH1-26 polypeptide directly to the carboxyl terminus of the Factor Xa cleavage site in the pMAL-c2 vector. A DNA fragment can be generated by PCR using SVPH1-26 DNA as the template DNA and two oligonucleotide primers. Use of the oligonucleotide primers generates a blunt-ended fragment of DNA that can be isolated by conventional means. This PCR product can be ligated together with pMAL-p2 (digested with the restriction endonuclease Xmn I) using conventional means. Positives clones can be identified by conventional means. Induction of expression and purification of the fusion protein can be performed as per the manufacturer's instructions. This construction facilitates a precise separation of the SVPH1-26 polypeptide from the fused maltose binding protein utilizing a simple protease treatment as per the manufacturer's instructions. In this manner, purified SVPH1-26 polypeptide can be obtained. Furthermore, such a constructed vector can be easily modified using known molecular biology techniques to generate additional fusion proteins.

[We could disclose the various expression vectors which were generated to express SVPH1-26 polypeptides. We should include the preferred method of expressing SVPH1-26 polypeptides known to the inventor.]

Another preferred embodiment of the invention is the use of SVPH1-26 polypeptides as molecular weight markers to estimate the apparent molecular weight of a sample protein by gel electrophoresis. An isolated and purified SVPH1-26 polypeptide molecular weight marker according to the invention has a molecular weight of approximately 81,548 Daltons in the absence of glycosylation. The SVPH1-26 polypeptide, together with a sample protein, can be resolved by denaturing polyacrylamide gel electrophoresis by conventional means (U. K. Laemmli, Nature 227:680-685, 1970) in two separate lanes of a gel containing sodium dodecyl sulfate and a concentration of acrylamide between 6-20%. Proteins on the gel can be visualized using a conventional staining procedure. The SVPH1-26 polypeptide molecular weight marker can be used as a molecular weight marker in the estimation of the apparent molecular weight of the sample protein. The unique amino acid sequence of SVPH1-26 (SEQ ID NO:2) specifies a molecular weight of approximately 81,548 Daltons. Therefore, the SVPH1-26 polypeptide molecular weight marker serves particularly well as a molecular weight marker for the estimation of the apparent molecular weight of sample proteins that have apparent molecular weights close to 81,548 Daltons. The use of this polypeptide molecular weight marker allows an increased accuracy in the determination of apparent molecular weight of proteins that have apparent molecular weights close to 81,548 Daltons. It is understood of course that many different techniques can be used for the determination of the molecular weight of a sample protein using SVPH1-26 polypeptides and that this embodiment in no way limits the scope of the invention.

Another preferred embodiment of the invention is the use of SVPH1-26 fragmented peptide molecular weight markers, generated by chemical fragmentation of SVPH1-26 polypeptide, as molecular weight markers to estimate the apparent molecular weight of a sample protein by gel electrophoresis. Isolated and purified SVPH1-26 polypeptide can be treated with cyanogen bromide under conventional conditions that result in fragmentation of the SVPH1-26 polypeptide molecular weight marker by specific hydrolysis on the carboxyl side of the methionine residues within the SVPH1-26 polypeptide (E. Gross, Methods in Enz. 11:238-255, 1967). Due to the unique amino acid sequence of the SVPH1-26 polypeptide, the fragmentation of SVPH1-26 polypeptide molecular weight markers with cyanogen bromide generates a unique set of SVPH1-26 fragmented peptide molecular weight markers. The distribution of methionine residues determines the number of amino acids in each peptide and the unique amino acid composition of each peptide determines its molecular weight.

The unique set of SVPH1-26 fragmented peptide molecular weight markers generated by treatment of SVPH1-26 polypeptide with cyanogen bromide comprises 14 fragmented peptides of at least 10 amino acids in size. The peptide encoded by amino acids 2-21 of SEQ ID NO:2 has a molecular weight of approximately 2,263 Daltons. The peptide encoded by amino acids 22-76 of SEQ ID NO:2 has a molecular weight of approximately 6,131 Daltons. The peptide encoded by amino acids 77-135 of SEQ ID NO:2 has a molecular weight of approximately 6,587 Daltons. The peptide encoded by amino acids 136-171 of SEQ ID NO:2 has a molecular weight of approximately 4,165 Daltons. The peptide encoded by amino acids 172-184 of SEQ ID NO:2 has a molecular weight of approximately 1,514 Daltons. The peptide encoded by amino acids 185-306 of SEQ ID NO:2 has a molecular weight of approximately 14,163 Daltons. The peptide encoded by amino acids 307-350 of SEQ ID NO:2 has a molecular weight of approximately 4,784 Daltons. The peptide encoded by amino acids 351-366 of SEQ ID NO:2 has a molecular weight of approximately 2,021 Daltons. The peptide encoded by amino acids 367-560 of SEQ ID NO:2 has a molecular weight of approximately 21,514 Daltons. The peptide encoded by amino acids 561-600 of SEQ ID NO:2 has a molecular weight of approximately 4,514 Daltons. The peptide encoded by amino acids 601-628 of SEQ ID NO:2 has a molecular weight of approximately 2,960 Daltons. The peptide encoded by amino acids 629-642 of SEQ ID NO:2 has a molecular weight of approximately 1,558 Daltons. The peptide encoded by amino acids 643-682 of SEQ ID NO:2 has a molecular weight of approximately 4,409 Daltons. The peptide encoded by amino acids 689-726 of SEQ ID NO:2 has a molecular weight of approximately 4,419 Daltons. Therefore, cleavage of the SVPH1-26 polypeptide by chemical treatment with cyanogen bromide generates a unique set of SVPH1-26 fragmented peptide molecular weight markers. The unique and known amino acid sequence of these SVPH1-26 fragmented peptides allows the determination of the molecular weight of these fragmented peptide molecular weight markers. In this particular case, SVPH1-26 fragmented peptide molecular weight markers have molecular weights of approximately 2,263; 6,131; 6,587; 4,165; 1,514; 14,163; 4,784; 2,021; 21,514; 4,514; 2,960; 1,558; 4,409; and 4,419 Daltons.

The SVPH1-26 fragmented peptide molecular weight markers, together with a sample protein, can be resolved by denaturing polyacrylamide gel electrophoresis by conventional means in two separate lanes of a gel containing sodium dodecyl sulfate and a concentration of acrylamide between 10-20%. Proteins on the gel can be visualized using a conventional staining procedure. The SVPH1-26 fragmented peptide molecular weight markers can be used as molecular weight markers in the estimation of the apparent molecular weight of the sample protein. The unique amino acid sequence of SVPH1-26 specifies a molecular weight of approximately 2,263; 6,131; 6,587; 4,165; 1,514; 14,163; 4,784; 2,021; 21,514; 4,514; 2,960; 1,558; 4,409; and 4,419 Daltons for the SVPH1-26 fragmented peptide molecular weight markers. Therefore, the SVPH1-26 fragmented peptide molecular weight markers serve particularly well as a molecular weight markers for the estimation of the apparent molecular weight of sample proteins that have apparent molecular weights close to 2,263; 6,131; 6,587; 4,165; 1,514; 14,163; 4,784; 2,021; 21,514; 4,514; 2,960; 1,558; 4,409; and 4,419 Daltons. Consequently, the use of these fragmented peptide molecular weight markers allows an increased accuracy in the determination of apparent molecular weight of proteins that have apparent molecular weights close to 2,263; 6,131; 6,587; 4,165; 1,514; 14,163; 4,784; 2,021; 21,514; 4,514; 2,960; 1,558; 4,409; and 4,419 Daltons.

In a further embodiment, the sample protein and the SVPH1-26 polypeptide can be simultaneously, but separately, treated with cyanogen bromide under conventional conditions that result in fragmentation of the sample protein and the SVPH1-26 polypeptide by specific hydrolysis on the carboxyl side of the methionine residues within the sample protein and the SVPH1-26 polypeptide. As described above, the SVPH1-26 fragmented peptide molecular weight markers generated by cleavage of the SVPH1-26 polypeptide with cyanogen bromide have molecular weights of approximately 2,263; 6,131; 6,587; 4,165; 1,514; 14,163; 4,784; 2,021; 21,514; 4,514; 2,960; 1,558; 4,409; and 4,419 Daltons.

The fragmented peptides from both the SVPH1-26 polypeptide and the sample protein can be resolved by denaturing polyacrylamide gel electrophoresis by conventional means in two separate lanes of a gel containing sodium dodecyl sulfate and a concentration of acrylamide between 10-20%. Fragmented peptides on the gel can be visualized using a conventional staining procedure. The SVPH1-26 fragmented peptide molecular weight markers can be used as molecular weight markers in the estimation of the apparent molecular weight of the fragmented proteins derived from the sample protein. As discussed above, the SVPH1-26 fragmented peptide molecular weight markers serve particularly well as a molecular weight markers for the estimation of the apparent molecular weight of fragmented peptides that have apparent molecular weights close to 2,263; 6,131; 6,587; 4,165; 1,514; 14,163; 4,784; 2,021; 21,514; 4,514; 2,960; 1,558; 4,409; and 4,419 Daltons. Consequently, the use of these SVPH1-26 fragmented peptide molecular weight markers allows an increased accuracy in the determination of apparent molecular weight of fragmented peptides that have apparent molecular weights close to 2,263; 6,131; 6,587; 4,165; 1,514; 14,163; 4,784; 2,021; 21,514; 4,514; 2,960; 1,558; 4,409; and 4,419 Daltons. The extent of fragmentation of the SVPH1-26 polypeptide is further used as a control to determine the conditions expected for complete fragmentation of the sample protein. It is understood of course that many chemicals could be used to fragment SVPH1-26 polypeptides and that this embodiment in no way limits the scope of the invention.

In another embodiment, unique sets of SVPH1-26 fragmented peptide molecular weight markers can be generated from SVPH1-26 polypeptide using enzymes that cleave the polypeptide at specific amino acid residues. Due to the unique nature of the amino acid sequence of the SVPH1-26 polypeptide, cleavage at different amino acid residues will result in the generation of different sets of fragmented peptide molecular weight markers.

An isolated and purified SVPH1-26 polypeptide can be treated with Achromobacter protease I under conventional conditions that result in fragmentation of the SVPH1-26 polypeptide by specific hydrolysis on the carboxyl side of the lysine residues within the SVPH1-26 polypeptide (T. Masaki et al., Biochim. Biophys. Acta 660:44-50, 1981; T. Masaki et al., Biochim. Biophys. Acta 660:51-55, 1981). Due to the unique amino acid sequence of the SVPH1-26 polypeptide, the fragmentation of SVPH1-26 polypeptide molecular weight markers with Achromobacter protease I generates a unique set of SVPH1-26 fragmented peptide molecular weight markers. The distribution of lysine residues determines the number of amino acids in each peptide and the unique amino acid composition of each peptide determines its molecular weight.

The unique set of SVPH1-26 fragmented peptide molecular weight markers generated by treatment of SVPH1-26 polypeptide with Achromobacter protease I comprises 22 fragmented peptides of at least 10 amino acids in size. The generation of 22 fragmented peptides with this enzyme treatment of the SVPH1-26 polypeptide, compared to 14 fragmented peptides with cyanogen bromide treatment of the SVPH1-26 polypeptide, clearly illustrates that both the size and number of the fragmented peptide molecular weight markers will vary depending upon the fragmentation treatment utilized to fragment the SVPH1-26 polypeptide. Both the size and number of these fragments are dictated by the amino acid sequence of the SVPH1-26 polypeptide.

The peptide encoded by amino acids 147 of SEQ ID NO:2 has a molecular weight of approximately 5,263 Daltons. The peptide encoded by amino acids 57-80 of SEQ ID NO:2 has a molecular weight of approximately 2,834 Daltons. The peptide encoded by amino acids 81-146 of SEQ ID NO:2 has a molecular weight of approximately 7,418 Daltons. The peptide encoded by amino acids 147-160 of SEQ ID NO:2 has a molecular weight of approximately 1,589 Daltons. The peptide encoded by amino acids 161-179 of SEQ ID NO:2 has a molecular weight of approximately 2,180 Daltons. The peptide encoded by amino acids 180-195 of SEQ ID NO:2 has a molecular weight of approximately 1,934 Daltons. The peptide encoded by amino acids 196-283 of SEQ ID NO:2 has a molecular weight of approximately 10,104 Daltons. The peptide encoded by amino acids 284-301 of SEQ ID NO:2 has a molecular weight of approximately 2,208 Daltons. The peptide encoded by amino acids 315-371 of SEQ ID NO:2 has a molecular weight of approximately 6,585 Daltons. The peptide encoded by amino acids 376-408 of SEQ ID NO:2 has a molecular weight of approximately 3,751 Daltons. The peptide encoded by amino acids 409-431 of SEQ ID NO:2 has a molecular weight of approximately 2,518 Daltons. The peptide encoded by amino acids 432-454 of SEQ ID NO:2 has a molecular weight of approximately 2,352 Daltons. The peptide encoded by amino acids 458-506 of SEQ ID NO:2 has a molecular weight of approximately 5,467 Daltons. The peptide encoded by amino acids 507-516 of SEQ ID NO:2 has a molecular weight of approximately 1,174 Daltons. The peptide encoded by amino acids 517-553 of SEQ ID NO:2 has a molecular weight of approximately 4,063 Daltons. The peptide encoded by amino acids 554-609 of SEQ ID NO:2 has a molecular weight of approximately 6,282 Daltons. The peptide encoded by amino acids 625-638 of SEQ ID NO:2 has a molecular weight of approximately 1,501 Daltons. The peptide encoded by amino acids 639-649 of SEQ ID NO:2 has a molecular weight of approximately 1,252 Daltons. The peptide encoded by amino acids 650-664 of SEQ ID NO:2 has a molecular weight of approximately 1,851 Daltons. The peptide encoded by amino acids 667-679 of SEQ ID NO:2 has a molecular weight of approximately 1,188 Daltons. The peptide encoded by amino acids 680-690 of SEQ ID NO:2 has a molecular weight of approximately 1,205 Daltons. The peptide encoded by amino acids 691-715 of SEQ ID NO:2 has a molecular weight of approximately 2,946 Daltons. Therefore, cleavage of the SVPH1-26 polypeptide by enzymatic treatment with Achromobacter protease I generates a unique set of SVPH1-26 fragmented peptide molecular weight markers. The unique and known amino acid sequence of these fragmented peptides allows the determination of the molecular weight of these SVPH1-26 fragmented peptide molecular weight markers. In this particular case, these SVPH1-26 fragmented peptide molecular weight markers have molecular weights of approximately 5,263; 2,834; 7,418; 1,589; 2,180; 1,934; 10,104; 2,208; 6,585; 3,751; 2,518; 2,352; 5,467; 1,174; 4,063; 6,282; 1,501; 1,252; 1,851; 1,188; 1,205; and 2,946 Daltons.

Once again, the SVPH1-26 fragmented peptide molecular weight markers, together with a sample protein, can be resolved by denaturing polyacrylamide gel electrophoresis by conventional means in two separate lanes of a gel containing sodium dodecyl sulfate and a concentration of acrylamide between 10-20%. Proteins on the gel can be visualized using a conventional staining procedure. The SVPH1-26 fragmented peptide molecular weight markers can be used as molecular weight markers in the estimation of the apparent molecular weight of the sample protein. The SVPH1-26 fragmented peptide molecular weight markers serve particularly well as a molecular weight markers for the estimation of the apparent molecular weight of proteins that have apparent molecular weights close to 5,263; 2,834; 7,418; 1,589; 2,180; 1,934; 10,104; 2,208; 6,585; 3,751; 2,518; 2,352; 5,467; 1,174; 4,063; 6,282; 1,501; 1,252; 1,851; 1,188; 1,205; and 2,946 Daltons. The use of these fragmented peptide molecular weight markers allows an increased accuracy in the determination of apparent molecular weight of proteins that have apparent molecular weights close to 5,263; 2,834; 7,418; 1,589; 2,180; 1,934; 10,104; 2,208; 6,585; 3,751; 2,518; 2,352; 5,467; 1,174; 4,063; 6,282; 1,501; 1,252; 1,851; 1,188; 1,205; and 2,946 Daltons.

In another embodiment, the sample protein and the SVPH1-26 polypeptide can be simultaneously, but separately, treated with Achromobacter protease I under conventional conditions that result in fragmentation of the sample protein and the SVPH1-26 polypeptide by specific hydrolysis on the carboxyl side of the lysine residues within the sample protein and the SVPH1-26 polypeptide. The SVPH1-26 fragmented peptide molecular weight markers and the fragmented peptides derived from the sample protein are resolved by denaturing polyacrylamide gel electrophoresis by conventional means in two separate lanes of a gel containing sodium dodecyl sulfate and a concentration of acrylamide between 10-20%. Fragmented peptides on the gel can be visualized using a conventional staining procedure. The SVPH1-26 fragmented peptide molecular weight markers can be used as molecular weight markers in the estimation of the apparent molecular weight of the sample protein. The SVPH1-26 fragmented peptide molecular weight markers serve particularly well as a molecular weight markers for the estimation of the apparent molecular weight of fragmented peptides that have apparent molecular weights close to 5,263; 2,834; 7,418; 1,589; 2,180; 1,934; 10,104; 2,208; 6,585; 3,751; 2,518; 2,352; 5,467; 1,174; 4,063; 6,282; 1,501; 1,252; 1,851; 1,188; 1,205; and 2,946 Daltons. The use of these SVPH1-26 fragmented peptide molecular weight markers allows an increased accuracy in the determination of apparent molecular weight of fragmented peptides that have apparent molecular weights close to 5,263; 2,834; 7,418; 1,589; 2,180; 1,934; 10,104; 2,208; 6,585; 3,751; 2,518; 2,352; 5,467; 1,174; 4,063; 6,282; 1,501; 1,252; 1,851; 1,188; 1,205; and 2,946 Daltons. The extent of fragmentation of the SVPH1-26 polypeptide is further used as a control to determine the conditions expected for complete fragmentation of the sample protein. It is understood of course that many enzymes could be used to fragment SVPH1-26 polypeptides and that this embodiment in no way limits the scope of the invention.

In another embodiment, monoclonal and polyclonal antibodies against SVPH1-26 polypeptides can be generated. Balb/c mice can be injected intraperitoneally on two occasions at 3 week intervals with 10 μg of isolated and purified SVPH1-26 polypeptide or peptides based on the amino acid sequence of SVPH1-26 polypeptides in the presence of RIBI adjuvant (RIBI Corp., Hamilton, Mont.). Mouse sera are then assayed by conventional dot blot technique or antibody capture (ABC) to determine which animal is best to fuse. Three weeks later, mice are given an intravenous boost of 3 μg of the SVPH1-26 polypeptide or peptides, suspended in sterile PBS. Three days later, mice are sacrificed and spleen cells fused with Ag8.653 myeloma cells (ATCC) following established protocols. Briefly, Ag8.653 cells are washed several times in serum-free media and fused to mouse spleen cells at a ratio of three spleen cells to one myeloma cell. The fusing agent is 50% PEG: 10% DMSO (Sigma). Fusion is plated out into twenty 96-well flat bottom plates (Corning) containing HAT supplemented DMEM media and allowed to grow for eight days. Supernatants from resultant hybridomas are collected and added to a 96-well plate for 60 minutes that is first coated with goat anti-mouse Ig. Following washes, ¹²⁵I-SVPH1-26 polypeptide or peptides are added to each well, incubated for 60 minutes at room temperature, and washed four times. Positive wells can be subsequently detected by autoradiography at −70° C. using Kodak X-Omat S film. Positive clones can be grown in bulk culture and supernatants are subsequently purified over a Protein A column (Pharmacia). It is understood of course that many techniques could be used to generate antibodies against SVPH1-26 polypeptides and fragmented peptides thereof and that this embodiment in no way limits the scope of the invention.

In another embodiment, antibodies generated against SVPH1-26 and fragmented peptides thereof can be used in combination with SVPH1-26 polypeptide or fragmented peptide molecular weight markers to enhance the accuracy in the use of these molecular weight markers to determine the apparent molecular weight and isoelectric point of a sample protein. SVPH1-26 polypeptide or fragmented peptide molecular weight markers can be mixed with a molar excess of a sample protein and the mixture can be resolved by two dimensional electrophoresis by conventional means. Polypeptides can be transferred to a suitable protein binding membrane, such as nitrocellulose, by conventional means.

Polypeptides on the membrane can be visualized using two different methods that allow a discrimination between the sample protein and the molecular weight markers. SVPH1-26 polypeptide or fragmented peptide molecular weight markers can be visualized using antibodies generated against these markers and conventional immunoblotting techniques. This detection is performed under conventional conditions that do not result in the detection of the sample protein. The sample protein is visualized using a conventional staining procedure. The molar excess of sample protein to SVPH1-26 polypeptide or fragmented peptide molecular weight markers is such that the conventional staining procedure predominantly detects the sample protein. The level of SVPH1-26 polypeptide or fragmented peptide molecular weight markers is such as to allow little or no detection of these markers by the conventional staining method. The preferred molar excess of sample protein to SVPH1-26 polypeptide molecular weight markers is between 2 and 100,000 fold. More preferably, the preferred molar excess of sample protein to SVPH1-26 polypeptide molecular weight markers is between 10 and 10,000 fold and especially between 100 and 1,000 fold.

The SVPH1-26 polypeptide or fragmented peptide molecular weight markers can be used as molecular weight and isoelectric point markers in the estimation of the apparent molecular weight and isoelectric point of the sample protein. The SVPH1-26 polypeptide or fragmented peptide molecular weight markers serve particularly well as molecular weight and isoelectric point markers for the estimation of apparent molecular weights and isoelectric points of sample proteins that have apparent molecular weights and isoelectric points close to that of the SVPH1-26 polypeptide or fragmented peptide molecular weight markers. The ability to simultaneously resolve the SVPH1-26 polypeptide or fragmented peptide molecular weight markers and the sample protein under identical conditions allows for increased accuracy in the determination of the apparent molecular weight and isoelectric point of the sample protein. This is of particular interest in techniques, such as two dimensional electrophoresis, where the nature of the procedure dictates that any markers should be resolved simultaneously with the sample protein.

In another embodiment, SVPH1-26 polypeptide or fragmented peptide molecular weight markers can be used as molecular weight and isoelectric point markers in the estimation of the apparent molecular weight and isoelectric point of fragmented peptides derived by treatment of a sample protein with a cleavage agent. It is understood of course that many techniques can be used for the determination of molecular weight and isoelectric point of a sample protein and fragmented peptides thereof using SVPH1-26 polypeptide molecular weight markers and peptide fragments thereof and that this embodiment in no way limits the scope of the invention.

SVPH1-26 polypeptide molecular weight markers encompassed by invention can have variable molecular weights, depending upon the host cell in which they are expressed. Glycosylation of SVPH1-26 polypeptide molecular weight markers and peptide fragments thereof in various cell types can result in variations of the molecular weight of these markers, depending upon the extent of modification. The size of SVPH1-26 polypeptide molecular weight markers can be most heterogeneous with fragments of SVPH1-26 polypeptide derived from the extracellular portion of the polypeptide. Consistent molecular weight markers can be obtained by using polypeptides derived entirely from the transmembrane and cytoplasmic regions, pretreating with N-glycanase to remove glycosylation, or expressing the polypeptides in bacterial hosts.

The interaction between SVPH1-26 and its counter-structure enables screening for small molecules that interfere with the SVPH1-26/SVPH1-26 counter-structure association and inhibit activity of SVPH1-26 or its counter-structure. For example, the yeast two-hybrid system developed at SUNY (described in U.S. Pat. No. 5,283,173 to Fields et al.) can be used to screen for inhibitors of SVPH1-26 as follows. SVPH1-26 and its counter-structure, or portions thereof responsible for their interaction, can be fused to the Gal4 DNA binding domain and Gal 4 transcriptional activation domain, respectively, and introduced into a strain that depends on Gal4 activity for growth on plates lacking histidine. Compounds that prevent growth can be screened in order to identify IL-1 inhibitors. Alternatively, the screen can be modified so that SVPH1-26/SVPH1-26 counter-structure interaction inhibits growth, so that inhibition of the interaction allows growth to occur. Another, in vitro, approach to screening for SVPH1-26 inhibition would be to immobilize one of the components (either SVPH1-26 or its counter-structure) in wells of a microtiter plate, and to couple an easily detected indicator to the other component. An inhibitor of the interaction is identified by the absence of the detectable indicator from the well.

In addition, SVPH1-26 polypeptides according to the invention are useful for the structure-based design of an SVPH1-26 inhibitor. Such a design would comprise the steps of determining the three-dimensional structure of such the SVPH1-26 polypeptide, analyzing the three-dimensional structure for the likely binding sites of substrates, synthesizing a molecule that incorporates a predictive reactive site, and determining the inhibiting activity of the molecule.

Antibodies immunoreactive with SVPH1-26 polypeptides, and in particular, monoclonal antibodies against SVPH1-26 polypeptides, are now made available through the invention. Such antibodies can be useful for inhibiting SVPH1-26 polypeptide activity in vivo and for detecting the presence of SVPH1-26 polypeptide in a sample.

As used herein, the term “SVPH1-26 polypeptides” refers to a genus of polypeptides that further encompasses proteins having the amino acid sequence 1-726 of SEQ ID NO:2, as well as those proteins having a high degree of similarity (at least 90% homology) with such amino acid sequences and which proteins are biologically active. In addition, SVPH1-26 polypeptides refers to the gene products of the nucleotides 1-726 of SEQ ID NO:2.

The isolated and purified SVPH1-26 polypeptide according to the invention has a molecular weight of approximately 81,548 Daltons in the absence of glycosylation. It is understood that the molecular weight of SVPH1-26 polypeptides can be varied by fusing additional peptide sequences to both the amino and carboxyl terminal ends of SVPH1-26 polypeptides. Fusions of additional peptide sequences at the amino and carboxyl terminal ends of SVPH1-26 polypeptides can be used to enhance expression of SVPH1-26 polypeptides or aid in the purification of the protein.

It is understood that fusions of additional peptide sequences at the amino and carboxyl terminal ends of SVPH1-26 polypeptides will alter some, but usually not all, of the fragmented peptides of SVPH1-26 polypeptides generated by enzymatic or chemical treatment.

It is understood that mutations can be introduced into SVPH1-26 polypeptides using routine and known techniques of molecular biology. It is further understood that a mutation can be designed so as to eliminate a site of proteolytic cleavage by a specific enzyme or a site of cleavage by a specific chemically induced fragmentation procedure. It is also understood that the elimination of the site will alter the peptide fingerprint of SVPH1-26 polypeptides upon fragmentation with the specific enzyme or chemical procedure.

The term “isolated and purified” as used herein, means that the SVPH1-26 polypeptide molecular weight markers or fragments thereof are essentially free of association with other proteins or polypeptides, for example, as a purification product of recombinant host cell culture or as a purified product from a non-recombinant source. The term “substantially purified” as used herein, refers to a mixture that contains SVPH1-26 polypeptide molecular weight markers or fragments thereof and is essentially free of association with other proteins or polypeptides, but for the presence of known proteins that can be removed using a specific antibody, and which substantially purified SVPH1-26 polypeptides or fragments thereof can be used as molecular weight markers. The term “purified” as referred to herein, refers to either the “isolated and purified” form of SVPH1-26 polypeptides or the “substantially purified” form of SVPH1-26 polypeptides, as both are described herein.

A “nucleotide sequence” refers to a polynucleotide molecule in the form of a separate fragment or as a component of a larger nucleic acid construct, that has been derived from DNA or RNA isolated at least once in substantially pure form (i.e., free of contaminating endogenous materials) and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

An SVPH1-26 polypeptide “variant” as referred to herein means a polypeptide substantially homologous to native SVPH1-26 polypeptides, but which has an amino acid sequence different from that of native SVPH1-26 polypeptides (human, murine or other mammalian species) because of one or more deletions, insertions or substitutions. The variant amino acid sequence preferably is at least 80% identical to a native SVPH1-26 polypeptide amino acid sequence, most preferably at least 90% identical. The percent identity can be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (I) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 351-2658, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring SVPH1-26 variants are also encompassed by the invention. Examples of such variants are proteins that result from alternate mRNA splicing events or from proteolytic cleavage of the SVPH1-26 polypeptides, wherein the SVPH1-26 proteolytic property is retained. Variations attributable to proteolysis include, for example, differences in the N- or C-termini upon expression in different types of host cells, due to proteolytic removal of one or more terminal amino acids from the SVPH1-26 polypeptides (generally from 1-5 terminal amino acids).

As stated above, the invention provides isolated and purified, or homogeneous, SVPH1-26 polypeptides, both recombinant and non-recombinant. Variants and derivatives of native SVPH1-26 polypeptides that can be used as molecular weight markers can be obtained by mutations of nucleotide sequences coding for native SVPH1-26 polypeptides. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making the alterations set forth above are disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

SVPH1-26 polypeptides can be modified to create SVPH1-26 polypeptide derivatives by forming covalent or aggregative conjugates with other chemical moieties, such as glycosyl groups, polyethylene glycol (PEG) groups, lipids, phosphate, acetyl groups and the like. Covalent derivatives of SVPH1-26 polypeptides can be prepared by linking the chemical moieties to functional groups on SVPH1-26 polypeptide amino acid side chains or at the N-terminus or C-terminus of a SVPH1-26 polypeptide or the extracellular domain thereof. Other derivatives of SVPH1-26 polypeptides within the scope of this invention include covalent or aggregative conjugates of SVPH1-26 polypeptides or peptide fragments with other proteins or polypeptides, such as by synthesis in recombinant culture as N-terminal or C-terminal fusions. For example, the conjugate can comprise a signal or leader polypeptide sequence (e.g. the α-factor leader of Saccharomyces) at the N-terminus of a SVPH1-26 polypeptide. The signal or leader peptide co-translationally or post-translationally directs transfer of the conjugate from its site of synthesis to a site inside or outside of the cell membrane or cell wall.

SVPH1-26 polypeptide conjugates can comprise peptides added to facilitate purification and identification of SVPH1-26 polypeptides. Such peptides include, for example, poly-His or the antigenic identification peptides described in U.S. Pat. No. 5,011,912 and in Hopp et al., Bio/Technology 6:1204, 1988.

The invention further includes SVPH1-26 polypeptides with or without associated native-pattern glycosylation. SVPH1-26 polypeptides expressed in yeast or mammalian expression systems (e.g., COS-1 or COS-7 cells) can be similar to or significantly different from a native SVPH1-26 polypeptide in molecular weight and glycosylation pattern, depending upon the choice of expression system. Expression of SVPH1-26 polypeptides in bacterial expression systems, such as E. coli, provides non-glycosylated molecules. Glycosyl groups can be removed through conventional methods, in particular those utilizing glycopeptidase. In general, glycosylated SVPH1-26 polypeptides can be incubated with a molar excess of glycopeptidase (Boehringer Mannheim).

Equivalent DNA constructs that encode various additions or substitutions of amino acid residues or sequences, or deletions of terminal or internal residues or sequences are encompassed by the invention. For example, N-glycosylation sites in the SVPH1-26 polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846, hereby incorporated by reference.

In another example, sequences encoding Cys residues that are not essential for biological activity can be altered to cause the Cys residues to be deleted or replaced with other amino acids, preventing formation of incorrect intramolecular disulfide bridges upon renaturation. Other equivalents are prepared by modification of adjacent dibasic amino acid residues to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding, or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

Nucleic acid sequences within the scope of the invention include isolated DNA and RNA sequences that hybridize to the native SVPH1-26 nucleotide sequences disclosed herein under conditions of moderate or severe stringency, and which encode SVPH1-26 polypeptides. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of about 60° C., 0.5×SSC, 0.1% SDS. Conditions of high stringency are defined as hybridization conditions as above, and with washing at 68° C., 0.2×SSC, 0.1% SDS. The skilled artisan will recognize that the temperature and wash solution salt concentration can be adjusted as necessary according to factors such as the length of the probe.

Due to the known degeneracy of the genetic code wherein more than one codon can encode the same amino acid, a DNA sequence can vary from that shown in SEQ ID NO:1 and still encode a SVPH1-26 polypeptide having the amino acid sequence of SEQ ID NO:2. Such variant DNA sequences can result from silent mutations (e.g., occurring during PCR amplification), or can be the product of deliberate mutagenesis of a native sequence.

The invention thus provides equivalent isolated DNA sequences encoding SVPH1-26 polypeptides, selected from: (a) DNA derived from the coding region of a native mammalian SVPH1-26 gene; (b) cDNA comprising the nucleotide sequence 1-2181 of SEQ ID NO:1; (c) DNA capable of hybridization to a DNA of (a) under conditions of moderate stringency and which encodes SVPH1-26 polypeptides; and (d) DNA which is degenerate as a result of the genetic code to a DNA defined in (a), (b) or (c) and which encodes SVPH1-26 polypeptides. SVPH1-26 polypeptides encoded by such DNA equivalent sequences are encompassed by the invention.

DNA that is equivalent to the DNA sequence of SEQ ID NO:1 will hybridize under moderately stringent conditions to the double-stranded native DNA sequence that encode polypeptides comprising amino acid sequences of 1-726 of SEQ ID NO:2. Examples of SVPH1-26 polypeptides encoded by such DNA, include, but are not limited to, SVPH1-26 polypeptide fragments and SVPH1-26 polypeptides comprising inactivated N-glycosylation site(s), inactivated protease processing site(s), or conservative amino acid substitution(s), as described above. SVPH1-26 polypeptides encoded by DNA derived from other mammalian species, wherein the DNA will hybridize to the complement of the DNA of SEQ ID NO:1 are also encompassed.

SVPH1-26 polypeptide-binding proteins, such as the anti-SVPH1-26 polypeptide antibodies of the invention, can be bound to a solid phase such as a column chromatography matrix or a similar substrate suitable for identifying, separating or purifying cells that express SVPH1-26 polypeptides on their surface. For example, the expression of SVPH1-26 in testis indicates that anti-SVPH1-26 polypeptide antibodies could be used to identify, separate, or purify testicular cells using conventional techniques. Adherence of SVPH1-26 polypeptide-binding proteins to a solid phase contacting surface can be accomplished by any means, for example, magnetic microspheres can be coated with SVPH1-26 polypeptide-binding proteins and held in the incubation vessel through a magnetic field. Suspensions of cell mixtures are contacted with the solid phase that has SVPH1-26 polypeptide-binding proteins thereon. Cells having SVPH1-26 polypeptides on their surface bind to the fixed SVPH1-26 polypeptide-binding protein and unbound cells then are washed away. This affinity-binding method is useful for purifying, screening or separating such SVPH1-26 polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner.

Alternatively, mixtures of cells suspected of containing SVPH1-26 polypeptide-expressing cells first can be incubated with a biotinylated SVPH1-26 polypeptide-binding protein. Incubation periods are typically at least one hour in duration to ensure sufficient binding to SVPH1-26 polypeptides. The resulting mixture then is passed through a column packed with avidin-coated beads, whereby the high affinity of biotin for avidin provides the binding of the SVPH1-26 polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

In the methods described above, suitable SVPH1-26 polypeptide-binding proteins are anti-SVPH1-26 polypeptide antibodies, and other proteins that are capable of high-affinity binding of SVPH1-26 polypeptides. A preferred SVPH1-26 polypeptide-binding protein is an anti-SVPH1-26 polypeptide monoclonal antibody.

SVPH1-26 polypeptides can exist as oligomers, such as covalently linked or non-covalently linked dimers or trimers. Oligomers can be linked by disulfide bonds formed between cysteine residues on different SVPH1-26 polypeptides. In one embodiment of the invention, a SVPH1-26 polypeptide dimer is created by fusing SVPH1-26 polypeptides to the Fc region of an antibody (e.g., IgG1) in a manner that does not interfere with biological activity of SVPH1-26 polypeptides. Example 2 is an example of such an embodiment. The Fc polypeptide preferably is fused to the C-terminus of a soluble SVPH1-26 polypeptide (comprising only the extracellular domain). General preparation of fusion proteins comprising heterologous polypeptides fused to various portions of antibody-derived polypeptides (including the Fc domain) has been described, e.g., by Ashkenazi et al. (PNAS USA 88:10535, 1991) and Byrn et al. (Nature 344:677, 1990), hereby incorporated by reference. A gene fusion encoding the SVPH1-26 polypeptide:Fc fusion protein is inserted into an appropriate expression vector. SVPH1-26 polypeptide:Fc fusion proteins are allowed to assemble much like antibody molecules, whereupon interchain disulfide bonds form between Fc polypeptides, yielding divalent SVPH1-26 polypeptides. If fusion proteins are made with both heavy and light chains of an antibody, it is possible to form a SVPH1-26 polypeptide oligomer with as many as four SVPH1-26 polypeptides extracellular regions. Alternatively, one can link two soluble SVPH1-26 polypeptide domains with a peptide linker.

Recombinant expression vectors containing a nucleic acid sequence encoding SVPH1-26 polypeptides can be prepared using well known methods. The expression vectors include a SVPH1-26 DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the SVPH1-26 DNA sequence. Thus, a promoter nucleotide sequence is operably linked to a SVPH1-26 DNA sequence if the promoter nucleotide sequence controls the transcription of the SVPH1-26 DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with SVPH1-26 polypeptides can be incorporated into expression vectors. For example, a DNA sequence for a signal peptide (secretory leader) can be fused in-frame to the SVPH1-26 nucleotide sequence so that the SVPH1-26 polypeptide is initially translated as a fusion protein comprising the signal peptide. A signal peptide that is functional in the intended host cells enhances extracellular secretion of the SVPH1-26 polypeptide. The signal peptide can be cleaved from the SVPH1-26 polypeptide upon secretion of SVPH1-26 polypeptide from the cell. In one embodiment of the invention, the Ig kappa signal sequence is us used. Example 2 is an example of such an embodiment.

Suitable host cells for expression of SVPH1-26 polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce SVPH1-26 polypeptides using RNAs derived from DNA constructs disclosed herein.

Prokaryotes include grain negative or gram positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a SVPH1-26 polypeptide can include an N-terminal methionine residue to facilitate expression of the recombinant polypeptide in the prokaryotic host cell. The N-terminal Met can be cleaved from the expressed recombinant SVPH1-26 polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct en expression vector using pBR322, an appropriate promoter and a SVPH1-26 DNA sequence are inserted into the pBR322 vector. Other commercially available vectors include, for example, pKK221-26 (Pharmacia Fine Chemicals, Uppsala, Sweden) and pGEM1 (Promega Biotec, Madison, Wis., USA). Other commercially available vectors include those that are specifically designed for the expression of proteins; these would include pMAL-p2 and pMAL-c2 vectors that are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982). A particularly useful prokaryotic host cell expression system employs a phage λP_{L} promoter and a cI1857ts thermolabile repressor sequence. Plasmid vectors available from the American Type Culture Collection, which incorporate derivatives of the λP_{L} promoter, include plasmid pHUB2 (resident in E. coli strain JMB9 (ATCC 37092)) and pPLc28 (resident in E. coli RR1 (ATCC 53082)).

SVPH1-26 may be cloned into the multiple cloning site of an ordinary bacterial expression vector. Ideally the vector would contain an inducible promoter upstream of the cloning site, such that addition of an inducer leads to high-level production of the recombinant protein at a time of the investigator's choosing. For some proteins, expression levels may be boosted by incorporation of codons encoding a fusion partner (such as hexahistidine) between the promoter and the gene of interest. The resulting “expression plasmid” may be propagated in a variety of strains of E. coli.

For expression of the recombinant protein, the bacterial cells are propagated in growth medium until reaching a pre-determined optical density. Expression of the recombinant protein is then induced, e.g. by addition of IPTG (isopropyl-b-D-thiogalactopyranoside), which activates expression of proteins from plasmids containing a lac operator/promoter. After induction (typically for 14 hours), the cells are harvested by pelleting in a centrifuge, e.g. at 5,000×G for 20 minutes at 4° C.

For recovery of the expressed protein, the pelleted cells may be resuspended in ten volumes of 50 mM Tris-HCl (pH 8)/1 M NaCl and then passed two or three times through a French press. Most highly-expressed recombinant proteins form insoluble aggregates known as inclusion bodies. Inclusion bodies can be purified away from the soluble proteins by pelleting in a centrifuge at 5,000×G for 20 minutes, 4° C. The inclusion body pellet is washed with 50 mM Tris-HCl (pH 8)/1% Triton X-100 and then dissolved in 50 mM Tris-HCl (pH 8)/8 M urea/0.1 M DTT. Any material that cannot be dissolved is removed by centrifugation (10,000×G for 20 minutes, 20° C.). The protein of interest will, in most cases, be the most abundant protein in the resulting clarified supernatant. This protein may be “refolded” into the active conformation by dialysis against 50 mM Tris-HCl (pH 8)/5 mM CaCl₂/5 mM Zn(OAc)₂/1 mM GSSG/0.1 mM GSH. After refolding, purification can be carried out by a variety of chromatographic methods such as ion exchange or gel filtration. In some protocols, initial purification may be carried out before refolding. As an example, hexahistidine-tagged fusion proteins may be partially purified on immobilized Nickel.

While the preceding purification and refolding procedure assumes that the protein is best recovered from inclusion bodies, those skilled in the art of protein purification will appreciate that many recombinant proteins are best purified out of the soluble fraction of cell lysates. In these cases, refolding is often not required, and purification by standard chromatographic methods can be carried out directly.

SVPH1-26 polypeptides alternatively can be expressed in yeast host cells, preferably from the Saccharomyces genus (e.g., S. cerevisiae). Other genera of yeast, such as Pichia, K. lactis, or Kluyveromyces, can also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980), or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli can be constructed by inserting DNA sequences from pBR322 for selection and replication in E. coli (Amp^{r} gene and origin of replication) into the above-described yeast vectors.

The yeast α-factor leader sequence can be employed to direct secretion of a SVPH1-26 polypeptide. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence can be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 μg/ml adenine, and 20 μg/ml uracil.

Yeast host cells transformed by vectors containing ADH2 promoter sequence can be grown for inducing expression in a “rich” medium. An example of a rich medium is one consisting of 1% yeast extract, 2% peptone, and 1% glucose supplemented with 80 μg/ml adenine and 80 μg/ml uracil. Derepression of the ADH2 promoter occurs when glucose is exhausted from the medium.

Mammalian or insect host cell culture systems could also be employed to express recombinant SVPH1-26 polypeptides. Baculovirus systems for production of heterologous proteins in insect cells are reviewed by Luckow and Summers, Bio/Technology 6:47 (1988). Established cell lines of mammalian origin also can be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CV1 (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine (Gibco/BRL) or Lipofectamine-Plus, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. Molecular Cloning: A Laboratory Manual, 2 ed. Vol. 1-3, Cold Spring Harbor Laboratory Press, 1989). Selection of stable transformants can be performed using resistance to cytotoxic drugs as a selection method. Kaufman et al., Meth. in Enzymology 185:487-511, 1990, describes several selection schemes, such as dihydrofolate reductase (DHFR) resistance. A suitable host strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (eg. DHFR) has been shown to improve transfectability of the host and expression of the heterologous cDNA (Kaufman, Meth in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in U.S. patent application Ser. No. 07/701,415, filed May 16, 1991, incorporated by reference herein. The vectors can be derived from retroviruses. In place of the native signal sequence, a heterologous signal sequence can be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type H IL-1 receptor signal peptide described in EP 460,846.

An isolated and purified SVPH1-26 polypeptide molecular weight marker according to the invention can be produced by recombinant expression systems as described above or purified from naturally occurring cells. SVPH1-26 polypeptides can be substantially purified, as indicated by a single protein band upon analysis by SDS-polyacrylamide gel electrophoresis (SDS-PAGE).

One process for producing SVPH1-26 polypeptides comprises culturing a host cell transformed with an expression vector comprising a DNA sequence that encodes a SVPH1-26 polypeptide under conditions sufficient to promote expression of the SVPH1-26 polypeptide. SVPH1-26 polypeptide is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify SVPH1-26 polypeptides. Some or all of the foregoing purification steps, in various combinations, are well known and can be employed to provide an isolated and purified recombinant protein.

It is possible to utilize an affinity column comprising a SVPH1-26 polypeptide-binding protein, such as a monoclonal antibody generated against SVPH1-26 polypeptides, to affinity-purify expressed SVPH1-26 polypeptides. SVPH1-26 polypeptides can be removed from an affinity column using conventional techniques, e.g., in a high salt elution buffer and then dialyzed into a lower salt buffer for use or by changing pH or other components depending on the affinity matrix utilized.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

Transformed yeast host cells are preferably employed to express SVPH1-26 polypeptides as secreted polypeptides in order to simplify purification. Secreted recombinant polypeptide from a yeast host cell fermentation can be purified by methods analogous to those disclosed by Urdal et al. (J. Chromatog. 296:171, 1984). Urdal et al. describe two sequential, reversed-phase HPLC steps for purification of recombinant human IL-2 on a preparative HPLC column.

SVPH1-26 polypeptide molecular weight markers can be analyzed by methods including sedimentation, gel electrophoresis, chromatography, and mass spectrometry. SVPH1-26 polypeptides can serve as molecular weight markers using such analysis techniques to assist in the determination of the molecular weight of a sample protein. A molecular weight determination of the sample protein assists in the identification of the sample protein.

SVPH1-26 polypeptides can be subjected to fragmentation into peptides by chemical and enzymatic means. Chemical fragmentation includes the use of cyanogen bromide to cleave under neutral or acidic conditions such that specific cleavage occurs at methionine residues (E. Gross, Methods in Enz. 11:238-255, 1967). This can further include further steps, such as a carboxymethylation step to convert cysteine residues to an unreactive species. Enzymatic fragmentation includes the use of a protease such as Asparaginylendopeptidase, Arginylendopeptidase, Achrombobacter protease I, Trypsin, Staphlococcus aureus V8 protease, Endoproteinase Asp-N, or Endoproteinase Lys-C under conventional conditions to result in cleavage at specific amino acid residues. Asparaginylendopeptidase can cleave specifically on the carboxyl side of the asparagine residues present within SVPH1-26 polypeptides. Arginylendopeptidase can cleave specifically on the carboxyl side of the arginine residues present within SVPH1-26 polypeptides. Achrombobacter protease I can cleave specifically on the carboxyl side of the lysine residues present within SVPH1-26 polypeptides (Sakiyama and Nakat, U.S. Pat. No. 5,248,599; T. Masaki et al., Biochim. Biophys. Acta 660:44-50, 1981; T. Masaki et al., Biochim. Biophys. Acta 660:51-55, 1981). Trypsin can cleave specifically on the carboxyl side of the arginine and lysine residues present within SVPH1-26 polypeptides. Staphlococcus aureus V8 protease can cleave specifically on the carboxyl side of the aspartic and glutamic acid residues present within SVPH1-26 polypeptides (D. W. Cleveland, J. Biol. Chem. 3:1102-1106, 1977). Endoproteinase Asp-N can cleave specifically on the amino side of the asparagine residues present within SVPH1-26 polypeptides. Endoproteinase Lys-C can cleave specifically on the carboxyl side of the lysine residues present within SVPH1-26 polypeptides. Other enzymatic and chemical treatments can likewise be used to specifically fragment SVPH1-26 polypeptides into a unique set of specific peptide molecular weight markers.

The resultant fragmented peptides can be analyzed by methods including sedimentation, electrophoresis, chromatography, and mass spectrometry. The fragmented peptides derived from SVPH1-26 polypeptides can serve as molecular weight markers using such techniques to assist in the determination of the molecular weight of a sample protein. Such a molecular weight determination assists in the identification of the sample protein. SVPH1-26 fragmented peptide molecular weight markers are preferably between 10 and 229 amino acids in size. More preferably, SVPH1-26 fragmented peptide molecular weight markers are between 10 and 100 amino acids in size. Even more preferable are SVPH1-26 fragmented peptide molecular weight markers between 10 and 50 amino acids in size and especially between 10 and 35 amino acids in size. Most preferable are SVPH1-26 fragmented peptide molecular weight markers between 10 and 20 amino acids in size.

Furthermore, analysis of the progressive fragmentation of SVPH1-26 polypeptides into specific peptides (D. W. Cleveland et al., J. Biol. Chem. 252:1102-1106, 1977), such as by altering the time or temperature of the fragmentation reaction, can be used as a control for the extent of cleavage of a sample protein. For example, cleavage of the same amount of SVPH1-26 polypeptide and sample protein under identical conditions can allow for a direct comparison of the extent of fragmentation. Conditions that result in the complete fragmentation of SVPH1-26 polypeptide can also result in complete fragmentation of the sample protein.

In addition, SVPH1-26 polypeptides and fragmented peptides thereof possess unique charge characteristics and, therefore, can serve as specific markers to assist in the determination of the isoelectric point of a sample protein or fragmented peptide using techniques such as isoelectric focusing. The technique of isoelectric focusing can be further combined with other techniques such as gel electrophoresis to simultaneously separate a protein on the basis of molecular weight and charge. An example of such a combination is that of two-dimensional electrophoresis (T. D. Brock and M. T. Madigan, Biology of Microorganisms 76-77 (Prentice Hall, 6d ed. 1991)). SVPH1-26 polypeptides and fragmented peptides thereof can be used in such analyses as markers to assist in the determination of both the isoelectric point and molecular weight of a sample protein or fragmented peptide.

Kits to aid in the determination of apparent molecular weight and isoelectric point of a sample protein can be assembled from SVPH1-26 polypeptides and peptide fragments thereof. Kits also serve to assess the degree of fragmentation of a sample protein. The constituents of such kits can be varied, but typically contain SVPH1-26 polypeptide and fragmented peptide molecular weight markers. Also, such kits can contain SVPH1-26 polypeptides wherein a site necessary for fragmentation has been removed. Furthermore, the kits can contain reagents for the specific cleavage of SVPH1-26 and the sample protein by chemical or enzymatic cleavage. Kits can further contain antibodies directed against SVPH1-26 polypeptides or fragments thereof.

Antisense or sense oligonucleotides comprising a single-stranded nucleic acid sequence (either RNA or DNA) capable of binding to a target SVPH1-26 mRNA sequence (forming a duplex) or to the SVPH1-26 sequence in the double-stranded DNA helix (forming a triple helix) can be made according to the invention. Antisense or sense oligonucleotides, according to the present invention, comprise a fragment of the coding region of SVPH1-26 cDNA (SEQ ID NO:1). Such a fragment generally comprises at least about 14 nucleotides, preferably from about 14 to about 30 nucleotides. The ability to create an antisense or a sense oligonucleotide, based upon a cDNA sequence for a given protein is described in, for example, Stein and Cohen, Cancer Res. 48:2659, 1988 and van der Krol et al., BioTechniques 6:958, 1988.

Binding of antisense or sense oligonucleotides to target nucleic acid sequences results in the formation of complexes that block translation (RNA) or transcription (DNA) by one of several means, including enhanced degradation of the duplexes, premature termination of transcription or translation, or by other means. The antisense oligonucleotides thus can be used to block expression of SVPH1-26 polypeptides. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation), but retain sequence specificity to be able to bind to target nucleotide sequences. Other examples of sense or antisense oligonucleotides include those oligonucleotides that are covalently linked to organic moieties, such as those described in WO 90/10448, and other moieties that increase affinity of the oligonucleotide for a target nucleic acid sequence, such as poly-(L-lysine). Further still, intercalating agents, such as ellipticine, and alkylating agents or metal complexes can be attached to sense or antisense oligonucleotides to modify binding specificities of the antisense or sense oligonucleotide for the target nucleotide sequence.

Antisense or sense oligonucleotides can be introduced into a cell containing the target nucleic acid sequence by any gene transfer method, including, for example, CaPO₄-mediated DNA transfection, electroporation, or by using gene transfer vectors such as Epstein-Barr virus. Antisense or sense oligonucleotides are preferably introduced into a cell containing the target nucleic acid sequence by insertion of the antisense or sense oligonucleotide into a suitable retroviral vector, then contacting the cell with the retrovirus vector containing the inserted sequence, either in vivo or ex vivo. Suitable retroviral vectors include, but are not limited to, the murine retrovirus M-MuLV, N2 (a retrovirus derived from M-MuLV), or the double copy vectors designated DCT5A, DCT5B and DCT5C (see PCT Application US 90/02656).

Sense or antisense oligonucleotides also can be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide can be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Isolated and purified SVPH1-26 polypeptides or a fragment thereof can also be useful itself as a therapeutic agent. SVPH1-26 polypeptides are introduced into the intracellular environment by well-known means, such as by encasing the protein in liposomes or coupling it to a monoclonal antibody targeted to a specific cell type.

SVPH1-26 DNA, SVPH1-26 polypeptides, and antibodies against SVPH1-26 polypeptides can be used as reagents in a variety of research protocols. A sample of such research protocols are given in Sambrook et al. Molecular Cloning: A Laboratory Manual, 2 ed. Vol. 1-3, Cold Spring Harbor Laboratory Press, (1989).

For example, these reagents can serve as markers for cell specific or tissue specific expression of RNA or proteins. The expression of SVPH1-26 RNA only in testis indicates that the expression of SVPH1-26 RNA and polypeptides in testis derived cell lines or testicular tissues can be directly detected with the reagents of the invention. Therefore, these reagents can be used as markers for cell specific or tissue specific expression. Such markers can be used in the detection and purification of specific cell types, and in the analysis of various diseases associated with testis (Schmoll et al., Semin Oncol 25:174-185, 1998. Wahren et al., J. Natl Cancer Inst 58:489-98; 1977; Beckstead, J. H., Am. J. Surg Pathol 7:341-9, 1983; Burke et al., Mod Pathol 1:475-479, 1988; Rajpert-De Meyts et al., Int J. Androl 17:85-92, 1994; Mead et al., J. Clin Oncol 10:85-94, 1992). In one embodiment, the identification of testicular cells in testicular biopsies by the reagents of the invention can facilitate the detection and prognosis of testicular cancers. For example, testis cells can be detected using probes of SVPH1-26 nucleic acid using conventional techniques, including Northern blots and in situ RNA hybridization (reviewed in Jin et al., J. Clin Lab Anal 11:2-9, 1997; McNicol et al, J. Pathol 182:250-261, 1997; Luke et al., Cell V is 5:49-53, 1998). It is understood of course that many different techniques can be used for the identification and purification of SVPH1-26 expressing cells and that this embodiment in no way limits the scope of the invention.

Similarly, these reagents can be used to investigate constitutive and transient expression of SVPH1-26 RNA or polypeptides. SVPH1-26 DNA can be used to determine the chromosomal location of SVPH1-26 DNA and to map genes in relation to this chromosomal location. SVPH1-26 DNA can also be used to examine genetic heterogeneity and heredity through the use of techniques such as genetic fingerprinting, as well as to identify risks associated with genetic disorders. SVPH1-26 DNA can be further used to identify additional genes related to SVPH1-26 DNA and to establish evolutionary trees based on the comparison of sequences. SVPH1-26 DNA and polypeptides can be used to select for those genes or proteins that are homologous to SVPH1-26 DNA or polypeptides, through positive screening procedures such as Southern blotting and immunoblotting and through negative screening procedures such as subtraction.

SVPH1-26 proteinase can be used as a reagent in analyses with other proteinases to compare the substrate specificity and activity of the proteinases. Chimeric proteinases can be generated by swapping fragments of SVPH1-26 proteinase with other proteinases. Such chimeric proteinases can be analyzed with respect to altered activity and specificity.

The proteinase activity of SVPH1-26 can be used as a detergent additive for the removal of stains having a protein component, similar to the use of proteases described in U.S. Pat. No. 5,599,400 and U.S. Pat. No. 5,650,315. The detergent composition can contain other known detergent constituents, such as surfactants, foam enhancers, fillers, enzyme stabilizers, chlorine bleach scavengers, other proteolytic enzymes, bacteriocides, dyes, perfumes, diluents, solvents, and other conventional ingredients. The detergent composition preferably contains between 0.001% to 10% SVPH1-26 proteinase. SVPH1-26 proteinase can be included in a detergent composition or can be combined with other constituents at the time of use as an additive. The detergent additive can be formulated as a liquid, powder, granulate, slurry, or other conventional form of a detergent additive.

SVPH1-26 polypeptides can also be used as a reagent to identify (a) any protein that SVPH1-26 polypeptide regulates, and (b) other proteins with which it might interact. SVPH1-26 polypeptides could be used by coupling recombinant protein to an affinity matrix, or by using them as a bait in the 2-hybrid system.

When used as a therapeutic agent, SVPH1-26 polypeptides can be formulated into pharmaceutical compositions according to known methods. SVPH1-26 polypeptides can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain SVPH1-26 polypeptides complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of SVPH1-26 polypeptides.

Within an aspect of the invention, SVPH1-26 polypeptides, and peptides based on the amino acid sequence of SVPH1-26, can be utilized to prepare antibodies that specifically bind to SVPH1-26 polypeptides. The term “antibodies” is meant to include polyclonal antibodies, monoclonal antibodies, fragments thereof such as F(ab′)2, and Fab fragments, as well as any recombinantly produced binding partners. Antibodies are defined to be specifically binding if they bind SVPH1-26 polypeptides with a Kₐ of greater than or equal to about 10⁷ M⁻¹. Affinities of binding partners or antibodies can be readily determined using conventional techniques, for example those described by Scatchard et al., Ann. N.Y Acad Sci., 51:660 (1949).

Polyclonal antibodies can be readily generated from a variety of sources, for example, horses, cows, goats, sheep, dogs, chickens, rabbits, mice, or rats, using procedures that are well-known in the art. In general, purified SVPH1-26 polypeptides, or a peptide based on the amino acid sequence of SVPH1-26 polypeptides that is appropriately conjugated, is administered to the host animal typically through parenteral injection. The immunogenicity of SVPH1-26 polypeptides can be enhanced through the use of an adjuvant, for example, Freund's complete or incomplete adjuvant. Following booster immunizations, small samples of serum are collected and tested for reactivity to SVPH1-26 polypeptides. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radio-immunoprecipitation, enzyme-linked immuno-sorbent assays (ELISA), dot blot assays, and sandwich assays, see U.S. Pat. Nos. 4,376,110 and 4,486,530.

Monoclonal antibodies can be readily prepared using well-known procedures, see for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKeam, and Bechtol (eds.), 1980. Briefly, the host animals, such as mice are injected intraperitoneally at least once, and preferably at least twice at about 3 week intervals with isolated and purified SVPH1-26 polypeptides or conjugated SVPH1-26 polypeptides, optionally in the presence of adjuvant. Mouse sera are then assayed by conventional dot blot technique or antibody capture (ABC) to determine which animal is best to fuse. Approximately two to three weeks later, the mice are given an intravenous boost of SVPH1-26 polypeptides or conjugated SVPH1-26 polypeptides. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as, ¹²⁵I-SVPH1-26 polypeptides is added to each well followed by incubation. Positive wells can be subsequently detected by autoradiography. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Other types of “antibodies” can be produced using the information provided herein in conjunction with the state of knowledge in the art. For example, antibodies that have been engineered to contain elements of human antibodies that are capable of specifically binding SVPH1-26 polypeptides are also encompassed by the invention.

Once isolated and purified, the antibodies against SVPH1-26 polypeptides can be used to detect the presence of SVPH1-26 polypeptides in a sample using established assay protocols. For example, antibodies against SVPH1-26 polypeptides can be used to detect or purify SVPH1-26 expressing cells, such as testis cells, by conventional techniques. Further, the antibodies of the invention can be used therapeutically to bind to SVPH1-26 polypeptides and inhibit its activity in vivo.

The purified SVPH1-26 polypeptides according to the invention will facilitate the discovery of inhibitors of SVPH1-26 polypeptides. The use of a purified SVPH1-26 polypeptide in the screening of potential inhibitors thereof is important and can eliminate or reduce the possibility of interfering reactions with contaminants.

In addition, SVPH1-26 polypeptides can be used for structure-based design of SVPH1-26 polypeptide-inhibitors. Such structure-based design is also known as “rational drug design.” The SVPH1-26 polypeptides can be three-dimensionally analyzed by, for example, X-ray crystallography, nuclear magnetic resonance or homology modeling, all of which are well-known methods. The use of SVPH1-26 polypeptide structural information in molecular modeling software systems to assist in inhibitor design and inhibitor-SVPH1-26 polypeptide interaction is also encompassed by the invention. Such computer-assisted modeling and drug design can utilize information such as chemical conformational analysis, electrostatic potential of the molecules, protein folding, etc. For example, most of the design of class-specific inhibitors of metalloproteases has focused on attempts to chelate or bind the catalytic zinc atom. Synthetic inhibitors are usually designed to contain a negatively-charged moiety to which is attached a series of other groups designed to fit the specificity pockets of the particular protease. A particular method of the invention comprises analyzing the three dimensional structure of SVPH1-26 polypeptides for likely binding sites of substrates, synthesizing a new molecule that incorporates a predictive reactive site, and assaying the new molecule as described above.

The specification is most thoroughly understood in light of the teachings of the references cited within the specification, which are hereby incorporated by reference. The embodiments within the specification and the following Examples provide an illustration of embodiments of the invention and should not be construed to limit the scope of the invention. The skilled artisan recognizes many other embodiments are encompassed by the claimed invention. In the following Examples, all methods described are conventional unless otherwise specified.

### EXAMPLE 1

### SVPH1-26 RNA Expression

Northern blots were purchased from Clonetech (catalog number 7760-1 and 7759-1, Palo Alto, Calif.) and contained RNA from tissues isolated from human heart, brain, placenta, lung, skeletal muscle, kidney, pancreas, spleen, thymus, prostate, testis, ovary, small intestine, colon, and peripheral blood leukocytes. The blot was pre-hybridized in Stark's buffer (50% formamide, 50 mM KP0₄, 5×SSC, 1% SDS, 5×Denhardt's, 0.05% sarcosyl, 300 μg/ml salmon sperm DNA) at 63° C. for at least 1 h and probed with a SVPH1-26 ³²P labeled riboprobe in Stark's buffer at 63° C. overnight (Cosman et al., Nature 312:768, 1984). Blots were then sequentially washed to high stringency (0.1×SSC, 0.1% SDS, 63° C.) and exposed to film (X-OMAT AR, Eastman Kodak Co., Rochester, N.Y.). Exposed films were developed in an automated x-ray film processor. The SVPH1-26 anti-sense riboprobe was prepared by in vitro transcription from a T7 RNA promoter with a commercially available kit (MAXIscript, Ambion, Inc., Austin, Tex.) using [α-³²P]UTP as the labeled nucleotide. Expression was detected only in testis.

### EXAMPLE 2

### SVPH1-26 Polypeptide Expression

DNA encoding the Ig kappa signal sequence was fused to the amino terminal end of the disintegrin domain of SVPH1-26 polypeptide (Cys410-Arg692 of SEQ ID NO:2). The carboxyl end of the disintegrin domain was fused to the Fc domain of IgG1 so that a Fc dimer fusion protein containing the disintegrin domain of SVPH1-26 polypeptide would be secreted out of the cell. The amino acid sequence of the fusion protein is as follows:

```
(SEQ ID NO:3)
    1 METDTLLLWV LLLWVPGSTG TSCGNLVVEE GEECDCGTIR QCAKDPCCLL
  51 NCTLHPGAAC AFGICCKDCK FLPSGTLCRQ QVGECDLPEW CNGTSHQCPD
101 DVYVQDGISC NVNAFCYEKT CNNHDIQCKE IFGQDARSAS QSCYQEINTQ
151 GNRFGHCGIV GTTYVKCWTP DIMCGRVQCE NVGVIPNLIE HSTVQQFHLN
201 DTTCWGTDYH LGMAIPDIGE VKDGTVCGPE IICIRKKCAS MVHLSQACQP
251 KTCNMRGICN NKQHCHCNHE WAPPYCKDKG YGGSADSGPP PKNNMEGLNV
301 MGKLRGSCDK THTCPPCPAP EAEGAPSVFL FPPKPKDTLM ISRTPEVTCV
351 VVDVSHEDPE VKFNWYVDGV EVHNAKTKPR EEQYNSTYRV VSVLTVLHQD
401 WLNGKEYKCK VSNKALPAPI EKTISKAKGQ PREPQVYTLP PSRDELTKNQ
451 VSLTCLVKGF YPSDIAVEWE SNGQPENNYK TTPPVLDSDG SFFLYSKLTV
501 DKSRWQQGNV FSCSVMHEAL HNHYTQKSLS LSPGK*
```

The Ig kappa signal sequence is encoded by amino acids 1-20 of SEQ ID NO:3. Amino acids 21 and 22 are spacer amino acids encoded by the restriction site used in the cloning. The disintegrin domain of SVPH1-26 polypeptide (Cys410-Arg692 of SEQ ID NO:2) is encoded by amino acids 23-305 of SEQ ID NO:3. Amino acids 306 and 307 are spacer amino acids encoded by the restriction site used in the cloning. The Fc domain of IgG1 is encoded by amino acids 308-535 of SEQ ID NO:3.

The DNA encoding this protein was inserted into the mammalian expression vector pDC412. pDC412 is a derivative of pDC406 (McMahan et al., EMBO J. 10:2821-2832 (1991)), and contains the same expression elements. The expression vector was transfected into COS cells, as described by Cosman et al., Nature 312:768, 1984. The protein was expressed and media containing the fusion protein was collected. The Fc fission protein was purified by standard methods, as described in Goodwin et al., Cell 73:447-456 (1993). This protein can be used to identify its counter-structure and to find inhibitors of this binding.

What is claimed is:

1. An isolated nucleic acid molecule selected from the group consisting of:
(a) a DNA that encodes a polypeptide comprising SEQ ID NO:2;
(b) DNA that encodes a fragment of the polypeptide of SEQ ID NO:2, wherein the fragment has proteinase activity;
(c) a DNA that hybridizes to either strand of a denatured, double-stranded DNA comprising the nucleic acid sequence of (a) or (b), wherein the hybridization conditions include 6×SSC, at 40° C. with washing conditions of 60° C., 0.5×SSC, 0.1% SDS, wherein the DNA encodes a polypeptide that has proteinase activity; and
(d) the DNA of SEQ ID NO:1.

2. An isolated nucleic acid molecule selected from the group consisting of:
(a) DNA that encodes a fragment of the polypeptide of SEQ ID NO:2, wherein the fragment has disintegrin activity; and
(b) a DNA that hybridizes to either strand of a denatured, double-stranded DNA comprising the nucleic acid sequence of (a), wherein the hybridization conditions include 6×SSC, at 40° C. with washing conditions of 60° C., 0.5×SSC, 0.1% SDS, wherein the DNA encodes a polypeptide that has disintegrin activity.

3. An isolated nucleic acid molecule that encodes a polypeptide comprising:
amino acids 1-27 of SEQ ID NO:2;
amino acids 28-197 of SEQ ID NO:2;
amino acids 198-398 of SEQ ID NO:2;
amino acids 399-502 of SEQ ID NO:2;
amino acids 503-692 of SEQ ID NO:2;
amino acids 693-714 of SEQ ID NO:2; or
amino acids 715-726 of SEQ ID NO:2.

4. A DNA the encodes a polypeptide that comprises an amino acid sequence that is at least 80% identical to SEQ ID NO:2, wherein the polypeptide has proteinase activity.

5. A DNA that encodes a polypeptide that comprises an amino acid sequence that is at least 80% identical to SEQ ID NO:2, wherein the polypeptide has disintegrin activity.

6. An expression vector comprising the DNA of any of claims 1-5.

7. A host cell comprising an expression vector of claim 6.

8. A polypeptide encoded by a DNA of any of claims 1-5.

9. A method for producing a polypeptide, the method comprising culturing a host cell of claim 7 under conditions that promote expression of the polypeptide.

10. The method of claim 9, further comprising recovering the polypeptide product.

11. An isolated antibody that binds to a polypeptide that consists of amino acids 1-726 of SEQ ID NO:2.

12. The antibody of claim 11, wherein the antibody is a monoclonal antibody.
